(19) United States
(12) Patent Application Publication
(10) Pub. No.: US 2025/0169818 A1
(43) Pub. Date: May 29, 2025

(54) WOUND CLOSURE SYSTEM AND METHOD

(71) Applicants: UNITY HEALTH TORONTO, TORONTO (CA); INVENTORR MD INC., TORONTO (CA)

(72) Inventors: JOÃO BAPTISTA DE REZENDE NETO, TORONTO (CA); ROBERT CHRISTOPHER BASS, TORONTO (CA)

(73) Assignees: UNITY HEALTH TORONTO, TORONTO, ON (CA); INVENTORR MD INC., TORONTO, ON (CA)

(21) Appl. No.: 18/840,570

(22) PCT Filed: Mar. 14, 2023
(86) PCT No.: PCT/CA2023/050326

§ 371 Date: Aug. 22, 2024

Related U.S. Application Data
(60) Provisional application No. 63/322,383, filed on Mar. 22, 2022.

(51) Int. Cl. A61B 17/08; A61B 17/00; A61B 90/00

(52) U.S. Cl. CPC A61B 17/08; A61B 2017/00557; A61B 2017/081; A61B 2090/064

(57) ABSTRACT

A wound management assembly includes a belt configured to partially wrap around the body of a patient in the area of a wound. First and second base members are configured to contact the patient on opposing sides of the wound and to grip opposing end regions of the belt to permit engagement, tensioning and detensioning of the belt relative to the base members. At least one cord is provided for connecting the base members to each other across the wound. The cord is adjustable to increase and decrease the pressure applied to the body of the patient. A pressure monitoring assembly is provided for monitoring the pressure applied to the body of the patient. The pressure monitoring assembly includes a bladder positionable between the at least one cord and the body of the patient, and a pressure gauge for displaying a pressure associated with the bladder.

### CROSS-REFERENCES TO RELATED APPLICATIONS

This application claims priority to U.S. Provisional Patent Application No. 63/322,383 filed on Mar. 22, 2022, which is incorporated herein by reference in its entirety.

### FIELD

The disclosure relates to medical devices, and in particular devices for the management of open wounds such as a post-surgery wound arising from abdominal surgery.

### BACKGROUND

International Patent Application Publication No. WO 2018/227292 A1 (McDonald et al.) discloses a wound management system including a belt configured to partially wrap around the body of a patient in the area of a wound; first and second base members configured to contact the patient on opposing sides of the wound; first and second clamp assemblies projecting from upper faces of the base members to grip opposing end regions of the belt, wherein at least one of the clamp assemblies is configured to releasably secure the belt to permit engagement, tensioning and detensioning of the belt relative to the base members; and at least one cord assembly for connecting the base members to each other across the wound. The cord assembly includes a cord, a cord anchor configured to attach a first end of the cord to a first of the base members and a cord retractor on the second of the base members for retracting the cord.

International Patent Application Publication No. WO 2016/127256 A1 (De Rezende Neto et al.) discloses a device for management of an open abdomen that includes a belt having first and second end portions. The belt is positionable to extend partially around a patient's torso in a taut configuration with the end portions positioned on opposed sides of an incision in the patient's abdomen. A first connector is at the first end portion and a second connector is at the second end portion. The connectors are positionable adjacent each other on the opposed sides of the incision. At least one cinching device is connected to and extends between the connectors. The cinching device is positionable to extend over the incision when the connectors are positioned adjacent each other on opposed sides of the incision. The cinching device is adjustable to cinch the end portions towards each other to hold the incision in an at least partially closed position.

### SUMMARY

The following summary is intended to introduce the reader to various aspects of the detailed description, but not to define or delimit any invention.

Wound management assemblies are disclosed.

According to some aspects, a wound management assembly includes a belt configured to partially wrap around the body of a patient in the area of a wound. First and second base members are configured to contact the patient on opposing sides of the wound and to grip opposing end regions of the belt to permit engagement, tensioning and detensioning of the belt relative to the base members. At least one cord is provided for connecting the base members to each other across the wound. The cord is adjustable to increase and decrease the pressure applied to the body of the patient. A pressure monitoring assembly is provided for monitoring the pressure applied to the body of the patient. The pressure monitoring assembly includes a bladder positionable between the at least one cord and the body of the patient, and a pressure gauge for displaying a pressure associated with the bladder, for monitoring of the pressure applied to the body of the patient.

The bladder may include an inflatable bag. When uninflated, the bladder may have a length of between about 5 inches and 15 inches and/or a width of between about 1 inch and about 3 inches. When inflated, the bladder may have a depth of between about 1 inch and about 1.5 inches.

The bladder may include fasteners for securing the bladder to the cord.

The pressure gauge may be configured to provide a pressure reading of between 0 inH₂O and 30 inH₂O. The pressure gauge may be coded to indicate a target pressure range to a user. The pressure gauge may be coded to indicate that the target pressure range is between about 20 inH₂O and 30 inH₂O. The pressure gauge may be coded to indicate that a pressure below 20 inH₂O is too low. The pressure gauge may be coded to indicate that a pressure above 27 inH₂O is too high.

The pressure gauge may be securable to the belt, the first base member, or the second base member. The pressure gauge may be securable to belt with a hook-and-loop fastener.

The assembly can further include a pump for inflating the bladder, or the bladder can be inflated by a fixed amount.

Methods for wound management are disclosed.

According to some aspects, a method for wound management includes: a. positioning first and second base members on a body of a patient on opposing sides of a wound; b. placing a bladder on the body of the patient between the first and second base members; c. positioning at least one cord over the bladder and connecting the base members to each other across the wound with the at least one cord; d. partially wrapping a belt around the body of the patient in an area of the wound and gripping opposing end regions of the belt with the first and second base members; and e. adjusting the at least one cord to increase or decrease the pressure applied to the body of the patient, whereby adjusting the at least one cord concurrently compresses or decompresses the bladder; and f. reading a pressure gauge associated with the bladder to assess the pressure applied to the body of the patient.

Step e. may include adjusting the at least one cord until the pressure gauge indicates that the pressure applied to the body of the patient is a target pressure.

Step e. may include adjusting the at least one cord until the pressure gauge indicates a pressure of between 20 inH₂O and 27 inH₂O.

The method may further include periodically repeating steps e. and f.

Step e. may include adjusting the at least one cord to increase the pressure applied to the body of the patient if the pressure gauge indicates a pressure of below 20 inH₂O, and adjusting the at least one cord to decrease the pressure applied to the body of the patient if the pressure gauge indicates a pressure of above 27 inH₂O.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example abdominal closure system, in which a belt is unlocked to permit adjustment;

FIG. 2 is a perspective view as in FIG. 1, showing the belt in the locked position against slippage relative to the base members;

FIG. 3 is a perspective view of base member of the system of FIGS. 1 and 2, showing the unlocked position;

FIG. 4 is partial cross-sectional view, in perspective, along line 4-4 of FIG. 3 showing components of the system and showing the cord assembly removed to show internal structures;

FIG. 5 is a partial cross-sectional view similar to FIG. 4, showing the cord assembly installed.

FIG. 6 is a further cross-sectional perspective view similar to FIG. 5, showing the clamp assembly in the fully disengaged position;

FIG. 7 is a side elevation view of the base member of FIGS. 1 to 6;

FIG. 8 is a perspective view of a portion of the system of FIGS. 1 to 7, namely opposing base members, positioned on opposed sides of a surgical wound of a patient;

FIG. 9A is a top plan view of a portion of the system of FIGS. 1 to 7, namely opposing base members which are positioned on opposed sides of a surgical wound of a patient.

FIG. 9B is a top plan view as in FIG. 9A, showing attachment of a belt and retraction of the system across a wound;

FIG. 10 is an end view of a portion of the system of FIGS. 1 to 7, showing the system being retracted across a surgical wound;

FIG. 11 is a side view of the system of FIGS. 1 to 7, showing the system in use on a patient.

FIG. 12 is end view similar to that of FIG. 10, showing the system in use on a patient;

FIG. 13 is a top plan view of the abdominal closure system of FIG. 1 in use on a patient with a pressure monitoring assembly; and

FIG. 14 is a perspective view of the pressure monitoring assembly of FIG. 13.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending on the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the phrase “at least one of A and B” is intended to mean only A (i.e. one or more A), only B (i.e. one or more B), or a combination of A and B.

Terms of degree such as “substantially”, “about”, and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

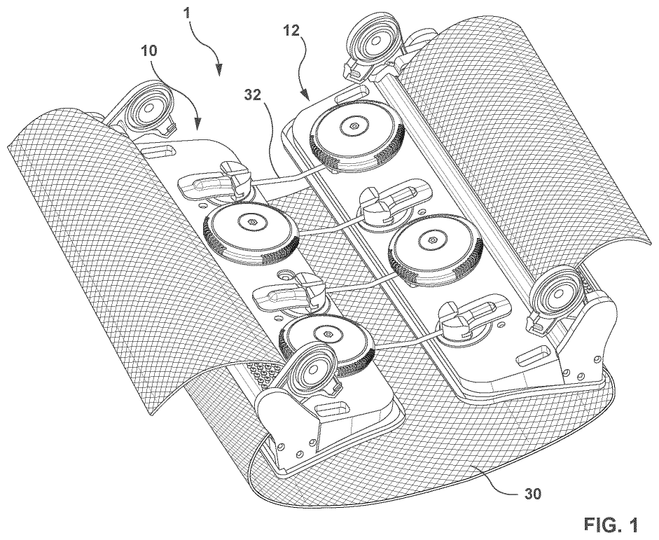

Referring first to FIGS. 1-7, an example abdominal closure system 1 is shown. The system 1 includes a pair of rigid base members 10 and 12, respectively, which are securable around a patient in the area of a wound, with a combination of a belt 30 and cords 32, described below. Base members 10 and 12 are identical to each other and interchangeable. Base members 10 and 12 include rectangular plates which can be fabricated from a rigid moulded or machined plastic such as ABS, Nylon, PVC, acetal and other polymers displaying similar physical properties.

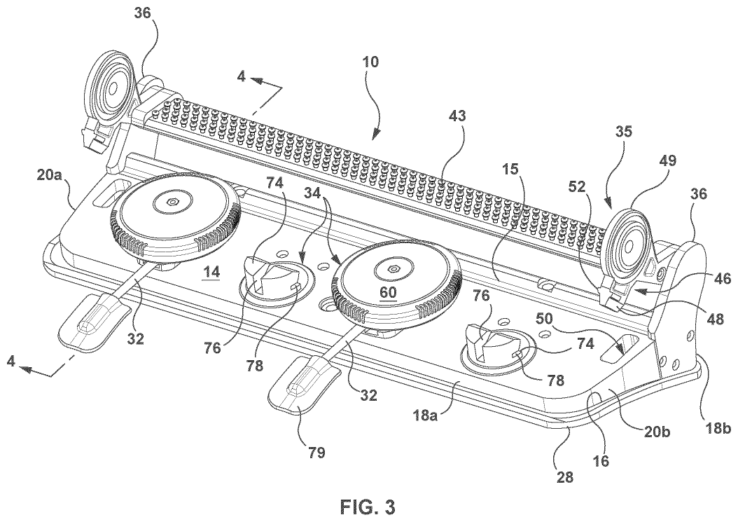

As shown in FIGS. 3 to 7, base members 10 and 12 each include an upper face 14, an opposing lower face 16, opposing lateral edges 18a and 18b and opposing end edges 20a and 20b. Base members 10 and 12 may be flat or curved to match the curvature of the midriff area of a typical patient. For reference herein, each base member has a longitudinal axis extending between end edges 20 and a lateral axis extending between lateral edges 18.

Example dimensions for base members 10 and 12 are between about 150 and 250 mm in length (longitudinal axis) and between about 30 and 100 mm in width (lateral axis). In one example, the length/width dimensions are about 225 mm×65 mm. Base members of these dimensions may be intended for use in treating normal adult patients undergoing major abdomen surgery. Different dimensions may be provided, depending on the application.

For reference and convenience of description, system 1 will be described herein with members 10 and 12 located in a horizontal position wherein the upper faces 14 are horizontal and face upwardly. When base members 10 and 12 are secured to a patient for managing a wound, as discussed below, lateral edges 18a of opposing members 10 and 12 face each other across a wound 24.

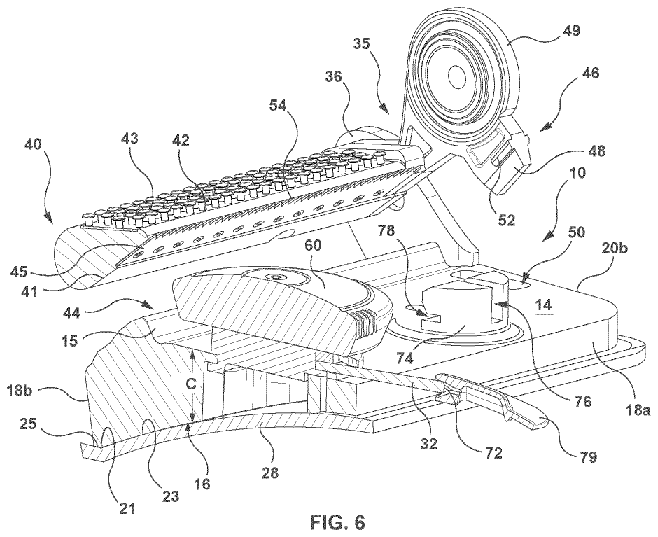

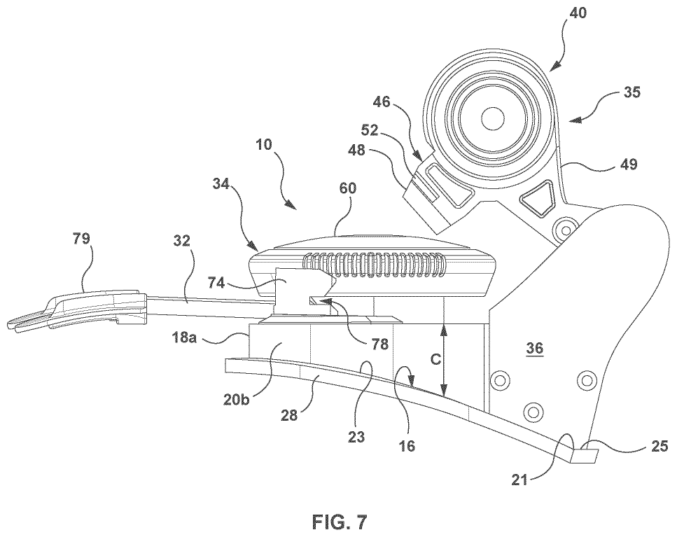

Referring to FIGS. 6 and 7, in the example shown, the upper face 14 of base members 10 and 12 has a flat region adjacent the inner lateral edge 18a (facing the opposing base member across the wound when in use), which is generally horizontal when in use. An outer portion of surface 14, adjacent outer lateral edge 18b, has a ridge that provides a sloping surface 15.

In the example shown, the lower faces 16 of members 10 and 12 are slightly curved to provide a trough-shaped, concave surface 23, in which the trough 23 extends along the longitudinal axis. Lower face 16 has an arcuate cross-sectional profile. The curvature of lower face 16 is configured to match the curvature of a typical human abdomen. The thickness of base members 10 and 12 (dimension C, seen in FIGS. 6 and 7) increases from first lateral edge 18a, which faces the opposing base member across the wound when in use, to the second lateral edge 18b, which faces outwardly from the wound. As seen in FIG. 7, base members 10 and 12 are generally tapered between lateral edges 18a and b, with the thin portion of the taper being located at the sides of the base members 10 and 12 that face each other across the wound. In use, this provides a generally horizontal upper surface 14 when the system 1 is positioned on a patient.

In the example shown, lower faces 16 are further characterized by a ridge 21 adjacent to outer later edge 18b. Ridge 21 defines an inflection line between the concavely-curved region of lower surface 16, and a flat, horizontally-projecting strip 25 that is adjacent to outer edge 18b. Ridge 21 provides additional traction against the patient's skin when the device is tensioned, to assist in drawing together the opposing edges of the wound 90.

In the example shown, the lower faces 16 of members 10 and 12 are each lined with a pad 28, which includes a soft, elastomeric and somewhat grippy material such as elastomeric silicon, having properties which make it suitable for contacting the patient's skin during a medical procedure. Preferably, pad 28 protrudes slightly beyond the edges 18 and 20 of members 10 and 12, to prevent direct contact between the patient and the rigid plastic material of members 10 and 12.

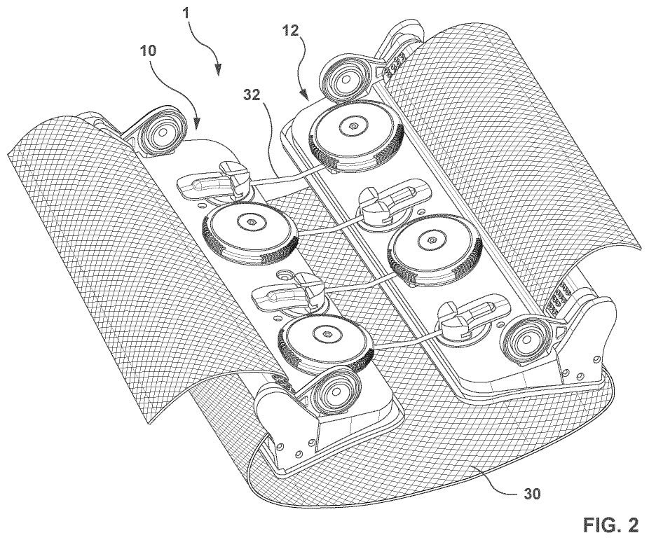

In the example shown, a flexible belt 30 is provided to secure base members 10 and 12 to the patient (see FIGS. 1 and 2). Belt 30 is configured to wrap partially around the patient to secure members 10 and 12 to the patient, in cooperation with cords 32 described below. As such, the combination of member 10 and 12, belt 30 and cords 32 fully encircle the patient, typically around the midriff region, when system 1 is engaged on a patient. Belt 30 may be tightened or loosened relative to members 10 and 12 to fit the device snugly to the patient. Belt 30 may be adjusted with respect to members 10 and 12 when members 10 and 12 are positioned on opposing sides of the wound, close to the margins of the wound, with belt 30 providing a snug fit around the patient. The method by which this may be performed is discussed below.

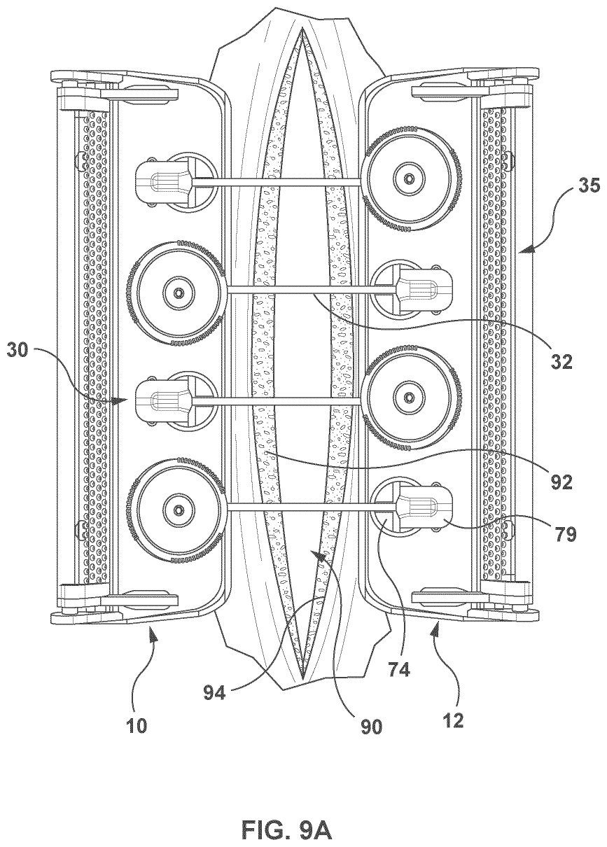

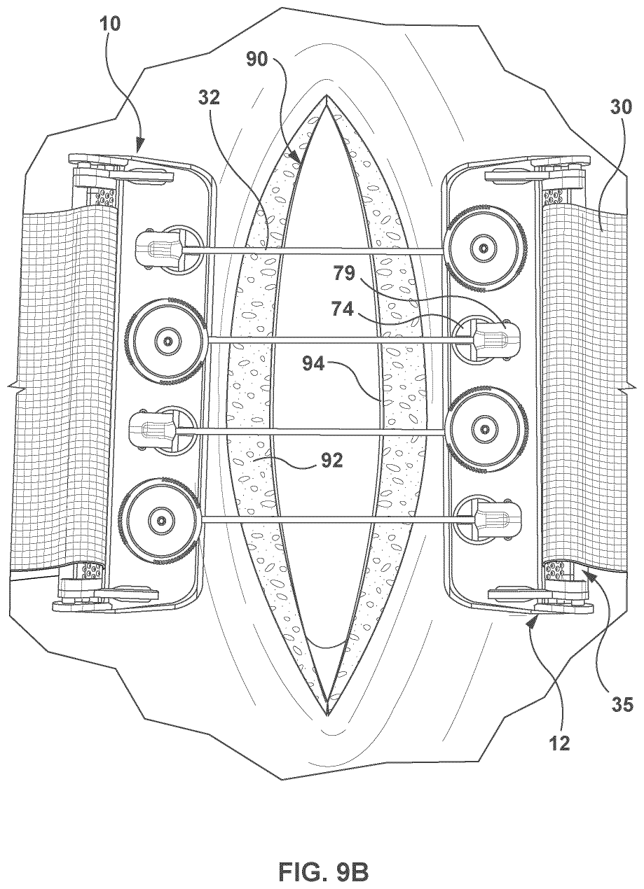

Belt 30 may be fabricated from a flexible material, such as mesh, woven fabric, flexible film or the like. Example materials include spandex, neoprene containing latex rubber, and similar fibers. Preferably, belt 30 is stretchable and elastic in at least its elongate dimension. The elasticity of belt 30 permits the device to retain a substantially consistent tension and a snug fit during patient muscle contractions, breathing and other movements by the patient. The degree of elasticity may be selected to provide a sufficiently tight fit to permit the device to accomplish the wound management results discussed below, while at the same time permitting a reasonably consistent tension during normal expansion and contraction of the patient's muscles and abdomen. For example, belt 30 may be configured to provide a maximum of tensile force in the range of 10-20 lbs linear force when secured around an average patient. Belt 30 is configured to wrap partially around a portion of the patient's body (such as the torso) out of contact with the wound area, to secure members 10 and 12 to the patient. At the wound area itself, members 10 and 12 are joined together with a retractable cord assembly 34, which may include multiple retractable cords 32, as discussed in more detail below. System 1 in its fully assembled form thus fully encircles the patient's torso. In particular, the patient is encircled with a combination of members 10 and 12, belt 30 and cords 32, as may be seen for example in FIG. 9.

Referring to FIGS. 3 to 7, at least one of base members 10 and 12, and optionally both of members 10 and 12, includes a clamp assembly 35 to releasably and adjustably secure belt 30 to members 10 and 12. Clamp assembly 35 engages belt 30 to the respective base member 10,12, and also permits belt 30 to be released from base members 10 and 12 and adjusted in effective length relative to one or both of base members 10 and 12.

In the example shown, clamp assembly 35 includes a pair of opposing, spaced apart tabs 36 that project upwardly from the upper faces 14 of base members 10 and 12. Tabs 36 are aligned with end edges 20 of members 10 and 12. Each tab 36 includes an inwardly-projecting horizontal post 38, and the posts 38 on opposing tabs 36 face each other. Posts 38 are co-axial and are aligned with the longitudinal axis. A releasable belt clamp bar 40 is rotatably journalled on posts 38. Clamp bar 40 is configured to releasably secure belt 30 to respective base members 10 and 12 by gripping belt 30 between clamp bar 30 and a portion of base member 10, 12. Clamp bar 40 extends substantially the length of base member 10 or 12 respectively, extending between the opposing end edges 20 thereof. Clamp bar 40 has post-holes (not shown) recessed into its end faces whereby it may rotate freely on posts 38.

In the example shown, clamp bar 40 rotates between a first, locked position, seen in FIG. 2, in which belt 30 is gripped between clamp bar 40 and sloping portion 15 of upper surface 14 and thus prevented from slipping, and a second, unlocked position, seen in FIGS. 1 and 3 to 7, in which clamp bar 40 is spaced from surface 14 and belt 30 can be adjusted or fully released from base member 10/12.

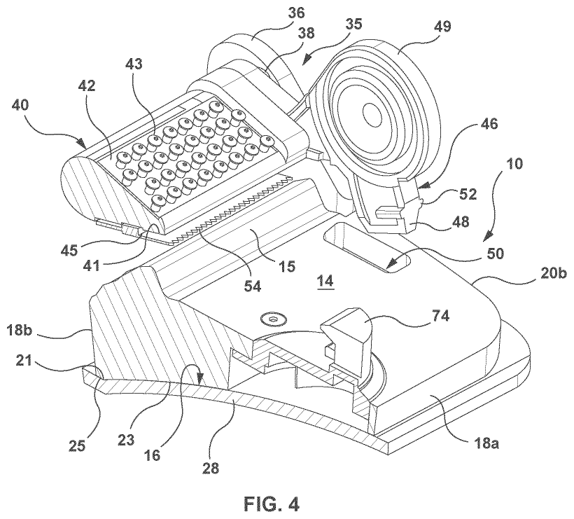

As seen in FIGS. 4-6, in the example shown, clamp bar 40 has an eccentric (non-circular) shape in cross section, which provides a cam-like function when rotated between the first and second positions. For this purpose, the axis of rotation of clamp bar 40 is offset from the central axis of the bar. Clamp bar 40 has a generally wing-shaped cross-sectional profile, characterized by a generally flat lower face 41 which is configured to contact the corresponding flat sloping portion 15 of base member 10/12. Clamp bar 40 and associated clamp assembly components are configured whereby lower face 41 of bar 40 is in contact and flush against upper face 14 of base member 10,12. When the respective faces are in contact, belt 30 is gripped between the respective flat surfaces 15 and 41. Clamp bar 40 further includes an upper flat face 42, opposed to lower face 41, which slopes upwardly. Upper face 42 is covered with an array of protrusions 43 or other surface which is adapted to grip the belt 30 (e.g. using a hook and loop fastener such as a Velcro™). This feature permits the free end of belt 30 to be wrapped around bar 40 and further secured to clamp bar 40, as seen in FIGS. 1 and 2. Lower face 41 of clamp bar 40 has a grip-plate 45 secured thereto, having a serrated edge 54 which engages belt 30 when clamp bar 40 is rotated into the grip position, as described below. In alternative examples, grip-plate 45 may be omitted.

When clamp bar 40 is rotated into the open position, as seen in FIG. 6, a gap 44 is exposed between clamp bar 40 and upper face 14 to permit belt 30 to slide through this gap.

In use, an end portion of belt 30 is passed through gap 44 and wrapped over clamp bar 40, and a portion of belt 30 is engaged to protrusions 43. Clamp bar 40 is then rotated into the closed position by gripping and rotating tabs 36. The cam-like shape of clamp bar 40 tends to urge clamp bar 40 further into the clamped position when belt 30 is retracted, thereby resisting slippage of belt 30 when this is retracted against base members 10 and 12.

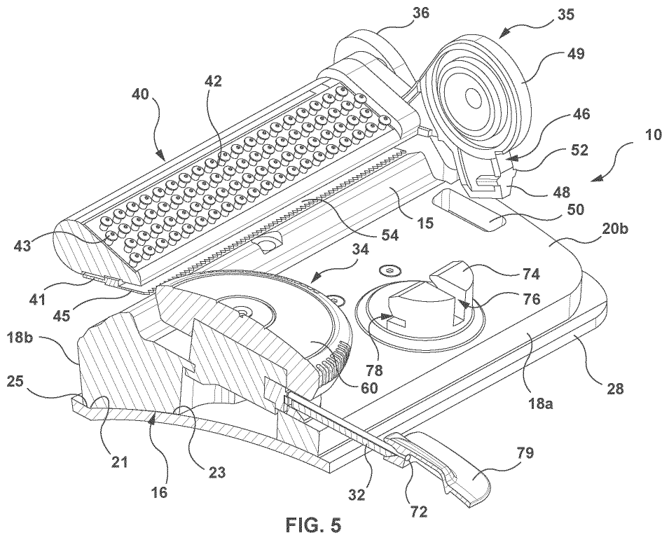

As seen in detail in FIGS. 5 and 7, in the example shown, clamp assembly 35 includes locking members 46 that lock clamp bar 40 to the corresponding member 10 or 12 into the locked position. Lock members 46 include a pair of opposing L-shaped projections extending from the opposing ends of clamp bar 40. Lock members 46 are attached to clamp bar 40 to rotate with it. Each of lock members includes a resilient plate 49 aligned with end edges 18, engaged at its proximal end to clamp bar 40. Plates 49 each have a disk-shaped portion at their proximal regions that have a slight depression to accommodate a user's finger, and opposing plates 49 may be squeezed together by the user. Plates 49 are configured to provide a suitable degree of resilience so the respective lock members may be squeezed together without too much difficulty, but not so easily as to permit inadvertent release during normal use. A flat projection 48 extends downwardly from the disc-shaped portion of plate 49 and is shaped to fit into a corresponding slot 50 within base member 10,12. Slots 50 are located adjacent end edges 20. Projections 48 are each provided with a laterally-projecting protrusion 52 at its distal end, such that opposing protrusions 52 project away from each other.

In the example shown, when clamp bar 40 is rotated towards the locked position, projections 48 of lock members 46 enter into slots 50. As protrusions 52 come into contact with the edges of slots 50, this urges projections 48 to flex towards each other to permit projections 48 to enter into slots 50. Projections 48 can thus snap-lock into slots 50 to lock clamp bar 40 into the locked position. Lock members 46 may afterwards be squeezed together to release lock members 46 from slot 50, thereby permitting rotation of clamp bars 40 into the unclamped position. This in turn disengages belt 30 from clamp assembly 35 to allow adjustment of belt 30 or its release from base member 10 or 12.

In the example shown, the serrated edge 54 of grip plate 45 serves to grip the belt 30 when in the clamped position. When clamp bar 40 is rotated away from the locked position, serrated edge 54 is out of contact with belt 30, thereby allowing belt 30 to slide through clamp assembly 35. However, as mentioned above, the grip plate 45 or the serrated edge 54 may be omitted.

Cord assembly 34 will now be described with specific reference to FIGS. 3-8.

In the example shown, cord assembly 34 includes multiple cord retractors, each of which includes a rotatable cord wind-up reel 60 which is independently and rotatably mounted to base plate 10 or 12. Reels 60 may include a commercially available take up reel of the type that provides a releasable ratchet mechanism to permit wind-up retraction of cord 32 by rotation of reel 60, and release of cord 60 by pulling upwardly or other manipulation of reel 60. Reels 60 are each mounted to an upwardly-projecting mount on base plates 10 and 12. In the present example, each of base members 10 and 12 is provided with two spaced apart reels 60, each configured to retract an individual one of cords 32. Reels 60 each include a spool surface for winding up a corresponding cord, and is capped with a gripper disk to allow the user to rotate the corresponding reel 60.

Cords 32 may be of monofilament style, braided and/or spiral styled constructions, and may be fabricated from a metal or polymer or a combination thereof. Examples include stainless steel, nylon, polyesters, PVC, and other materials.

Each of cords 32 has a free end 72 which is releasably anchored to a respective base member 10, 12 with a cleat 74. A corresponding reel 60 is mounted to an opposing base member to retract the opposing end of the cord. Cleat 74 may fixedly or releasably secured to free end 72; in the present example, cord 32 is releasably secured. Cleat 74 consists of an upwardly-projecting member, having a vertical slot 76 open to its upper surface and extending laterally. Cleat 74 also has a horizontal slot 78, open to the rear face of cleat 74. The free end 72 of cord 32 is fixed to a flat tab 79, which is configured to fit snugly within horizontal slot 78 to retain cord 32 to cleat 74 in a position in which cord 32 is inserted within vertical slot. The tension applied to cord 32 when device 1 is secured to a patient retains tab 79 within corresponding slot 78. In this fashion, the free end 72 of cord 32 is securely anchored to one of base members 10 or 12 when in use, with the other end of cord 32 being engaged to reel 60 on the other one of base members 10 or 12.

In the illustrated example, four cords 32 are provided, each associated with a corresponding wind up reel 60 and cleat 74 for independently anchoring the respective ends of each cord 32. The reels 60 and cleats 74 are staggered, whereby each of members 10 and 12 is provided with two cleats and two reels, arranged an alternating fashion. A different configuration and number of such cords 32 and anchoring cleats may be provided, depending on the overall size of base members 10 and 12 and other considerations.

System 1 may be provided within a range of dimensions of its components. For example, base members 10 and 12 may be approximately 225×65 mm in length x width to accommodate an average sized adult. In other examples, the base members may be outside of this range in versions scaled to accommodate above or below-average sized persons. The band may be dimensioned to accommodate a range of adult circumferences of between 75 cm and 150 cm but may fall outside of that range in versions scaled to accommodate above or below average sized persons. The cords 32 may be about 45 mm in length, which permits a broad range of extension and wind up inside of the reel.

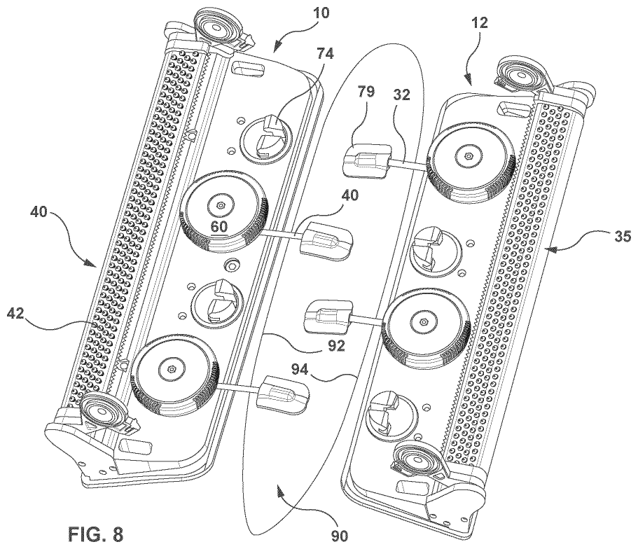

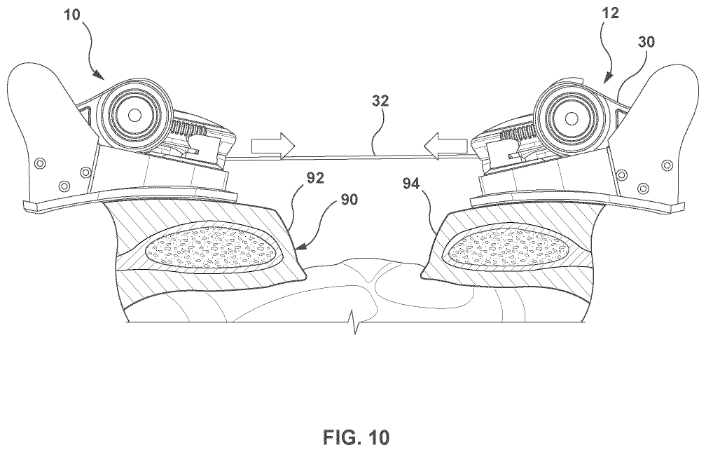

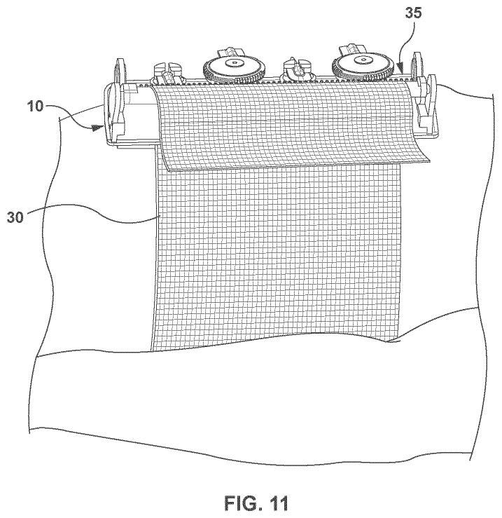

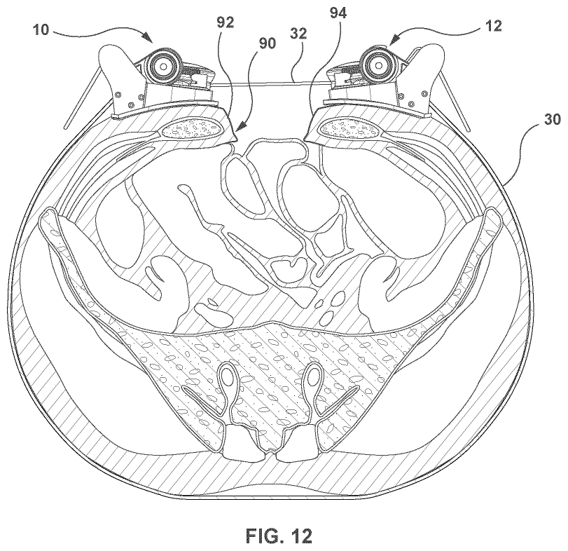

A method of using system 1 to manage a wound 90 is shown in FIGS. 8-12. Wound 90 may be an abdominal wound, such as a post-surgical wound as shown in FIGS. 9A, 9B and 10, which extends longitudinally (i.e. head to toe direction) along the midline of the patient's abdomen. Wound 90 is defined by wound margins 92 and 94. System 1 may be supplied to the health practitioner with belt 30 disengaged from one or both of base members 10 and 12, and cords 32 disengaged from cleats 74. One end of belt 30 may then be engaged with one of base members 10 and 12. Base members 10 and 12 are then positioned adjacent the margins of the wound, as seen in the Figures. In this example, base members 10 and 12 are positioned on the rectus muscles of the abdomen. The tensioning effect of device 1 provides a splinting effect that acts against the naturally occurring wound-spreading effect.

Typically, lateral edges 18 will be slightly displaced from the exposed edges of wound 90 to prevent the risk of contact of the base members 10 and 12 with the open wound edge. The free (unattached) ends of cords 32 are then anchored to corresponding cleats with tabs 79, so that cords 32 extend across the wound and base members 10 and 12 are prevented from further separation from each other. Cords 32 preferably remain spaced above wound 90 at all times during application, adjustment and use of the device, to avoid contact between wound 90 and cords 32.

At this point, belt 30 may be placed around the patient's body. For example, if wound 90 is on the dorsal midriff (belly) side of the patient, as shown in the Figures, belt 30 will be passed around the back (dorsal) midriff of the patient. The free ends of belt 30 are then engaged to the clamp assemblies 35 of the respective base members 10 and 12. Belt 30 is then retracted to provide a snug fit of device 1 to the patient. As discussed above, the elastic properties of belt 30 permits base members 10 and 12 to remain in their original positions without shifting as the patient breathes and other muscle movements.

Cords 32 may be periodically retracted to draw the wound edges together. For example, cord retractions may be performed in a series of discrete retractions performed in a staged fashion 2-3 times daily for several days following surgery, to fully close wound 90. In other examples, discrete retractions of cords 32 are performed hourly, 12× daily, 6× daily, 3× daily, 2× daily or daily. The system 1 may, for example, be used for treatment of the patient over a period of ½ day, 1 day, 2 days, 3 days, 4 days, 5 days, 6 days or 7 days. The device may be secured to the patient for the entire course of treatment. Adjustments of cords 32 may typically be performed as fine adjustments, for example about 5 mm of retraction per discrete retraction stage. This may range to a gross adjustment of about 100 mm. For example, the gross adjustment of cords 32 may be performed during the initial accommodation phase and the magnitude of the adjustments may then decrease over time. Patients may be monitored for intra-abdominal pressure (as described below) and this characteristic would supersede any linear measurement targets, whereby the amount of retraction that is performed, as well as the timing of stages, is adjusted based on patient response as determined by individual monitoring of the patient.

Retraction of the cords 32 to close a wound 90 is shown schematically in FIGS. 9B and 10. Retraction of cords 32 may prevent the loss of domain and facilitate closure, in that it engages oblique and a transverse muscles of the abdomen, creating an opposing force to the retraction and lateralization of the abdominal wall that would otherwise tend to separate the wound edges thereby preventing healing. When the system is used as described herein, the discrete retractions of the cords 32 delivers gradual traction of the abdominal muscles towards the midline. This action prevents lateralization of the abdominal wall muscles and brings the wound edges towards the midline. Furthermore, use of the system 1 as described herein serves to engage oblique and transverse muscles, thereby creating an opposing force against retraction and lateralization of the abdominal wall.

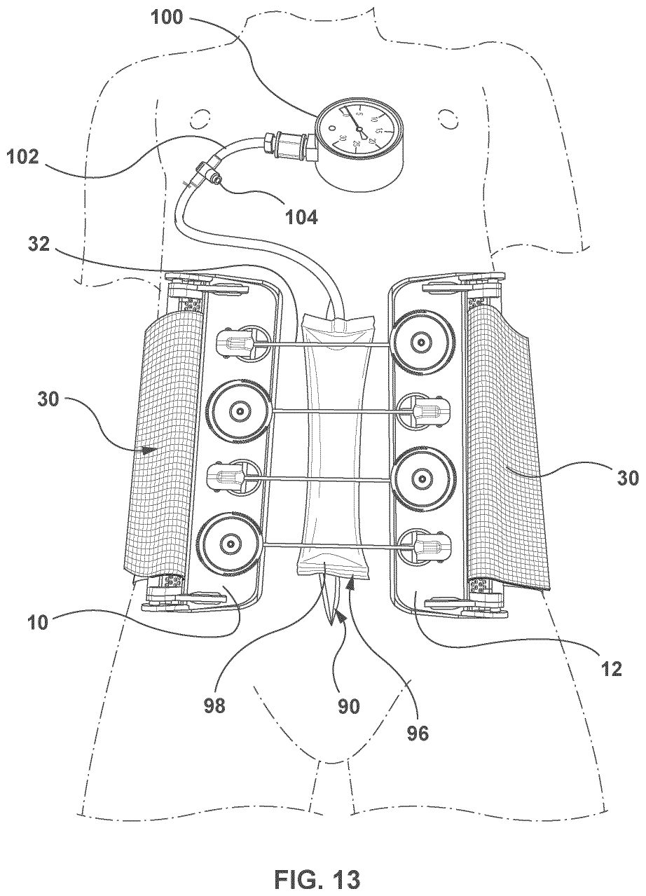

Referring now to FIG. 13, an example is shown in which the system 1 is used with a pressure monitoring assembly 96 that monitors the pressure applied to the patient (i.e. the system 1 and pressure monitoring assembly 96 together form a wound management assembly). Particularly, the pressure monitoring assembly 96 can monitor the pressure between the cords 32 (only one of which is labelled) and patient's body. This can allow for the cords 32 to be adjusted in response to the needs of the patient, and can, for example minimize the risk of abdominal compartment syndrome. For example, if the pressure reading from the pressure monitoring assembly 96 is too high, the cords 32 can be loosened in order to avoid or minimize the risk of abdominal compartment syndrome. Alternatively, if the pressure reading is too low, the cords 32 can be tightened to promote healing.

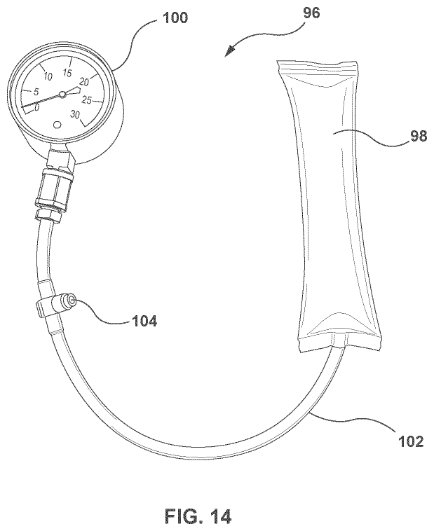

Referring to FIG. 14, the pressure monitoring assembly 96 is shown in greater detail. In the example shown, the pressure monitoring assembly includes a bladder 98 in the form of an inflatable bag. In the example shown, the bladder is shaped and sized to sit between and contact the cords 32 and the patient's body, and has a length similar to that of the base members 10 and 12. That is, the bladder 98 is generally elongate, and when uninflated, has a length of between about 5 inches and 15 inches (e.g. about 10 inches) and a width of between about 1 inch and 3 inches (e.g. about 2.125 inches). When inflated, the bladder has a depth of between about 1 inch and 1.5 inches (e.g. about 1.25 inches). Optionally, the bladder 98 can be provided with clips (not shown) or other fasteners for securing the bladder to the cords 32, to prevent movement of the bladder 98 in use. The bladder may be made, for example, from medical grade polyurethane.

Referring still to FIG. 14, the pressure monitoring assembly 96 further includes a pressure gauge 100, which is connected to the bladder 98 by a line 102, and which displays a pressure associated with the bladder 98. The pressure gauge 100 can, for example, provide a pressure reading of between about 0 inH₂O (i.e. about 0 mmHg) and about 30 inH₂O (i.e. about 56 mmHg). Optionally, the pressure gauge 100 can be coded (e.g. color coded) to indicate a target pressure range to the user. For example, the section of the pressure gauge between about 20 inH₂O (i.e. about 37 mmHg) and about 27 inH₂O (i.e. about 50 mmHg) can be colored green to indicate to the user that this is a target pressure. The section below about 20 inH₂O (i.e. about 37 mmHg) can be colored yellow to indicate to the user that the pressure is too low, and the section above about 27 inH₂O (i.e. about 50 mmHg) can be colored red to indicate to the user that the pressure is too high. Optionally, in use, the pressure gauge 98 can rest on the body of the patient, or can be secured to the system 1. For example, the pressure gauge 98 can be provided with a fastener such as a hook and loop fastener (e.g. Velcro™) backing for securing the pressure gauge 98 to the belt 30, the first base member, or the second base member.

In use, the bladder 98 may be provided pre-inflated, for example with air or another gas or a liquid. The bladder 98 may be pre-inflated to a fixed amount, so that any further pressure applied to the bladder will result in a reading on the pressure gauge. For example, the bladder 98 may be pre-inflated so that the pressure gauge reads slightly higher than zero (e.g. about 1 inH₂O (i.e. about 2 mmHg), or about 2 inH₂O (i.e. about 4 mmHg), or about 5 in H₂O (i.e. about 9 mmHg)). Alternatively, the pressure monitoring assembly 96 may be provided with a pump (not shown) for inflating the bladder 98 via port 104, together with instructions to inflate the bladder 98 until the pressure gauge reads slightly higher than zero. Optionally, the pressure gauge 100 can be coded (e.g. color coded) to indicate a target initial pressure range to the user. For example, the section of the pressure gauge below about 5 inH₂O (i.e. about 9 mmHg) can be colored blue to indicate to the user that this should be the reading prior to use of the bladder 98.

Referring back to FIG. 13, in use, after the base members 10 and 12 are positioned on a patient's body adjacent the margins of the wound and on oppose sides of the wound, and prior to attaching the ends of the cords 32 to the cleats (not labelled in FIG. 13), the bladder 98 may be placed on the patient's body, in close proximity to the wound (preferably directly on top of the wound), and between the first and second base members. The bladder may be pre-inflated prior to placement, or may be inflated after placement. The cords 32 may then be positioned over the bladder and the ends of the cords 32 may then be attached to the cleats, to connect the base members 10 and 12 to each other across the wound. The belt 30 may then be partially wrapped around the patient's body in the area of the wound and opposed end regions of the belt may be gripped with the base members 10 and 12 (e.g. engaged to the clamp assemblies (not labelled in FIG. 13) of the respective base members 10 and 12). The cords 32 may then be adjusted, for example retracted to increase the pressure applied to the body of the patient, or loosened to decrease the pressure applied to the body of the patient. As the cords 32 are adjusted, the pressure gauge 100 can be read in order to assess the pressure applied to the body of the patient. In the case of retraction, as the cords 32 retract, the bladder 98 will be compressed between the cords 32 and the patient's body, and the reading on the pressure gauge 100 will increase. The cords 32 can continue to be retracted until the pressure gauge 100 indicates that a target pressure (e.g. a pressure of between about 20 in H₂O and about 27 in H₂O (i.e. between about 37 mmHg and about 50 mmHg), e.g. about 23 inH₂O (i.e. about 43 mmHg)) has been reached. In the case of loosening, as the cords 32 are loosened, the bladder 98 will be decompressed between the cords 32 and the patient's body, and the reading on the pressure gauge 100 will decrease. The cords 32 can continue to be loosened until the pressure gauge 100 indicates that the target pressure has been reached. As noted above, the system 1 may be used for treatment of the patient over a period of ½ day, 1 day, 2 days, 3 days, 4 days, 5 days, 6 days, 7 days, or more. During this course of treatment, the steps of adjusting the cords 32 and reading the pressure gauge 100 may be repeated. That is, the cords 32 may be periodically adjusted in response to the reading on the pressure gauge 100. For example, as healing continues, the pressure reading may drop to below 20 inH₂O (i.e. about 37 mmHg), and thus the cords 32 can be further adjusted (i.e. retracted) to increase the pressure. Alternatively, if the patient suffers from complications, the pressure reading may increase to above 27 inH₂O (i.e. about 50 mmHg), and thus the cords 32 can be further adjusted (i.e. loosened) to decrease the pressure.

The pressure monitoring assembly 96 may further be used with other wound management devices, such as the device disclosed in U.S. Pat. No. 10,524,793 (to De Rezende Neto) issued on Jan. 7, 2020, which is incorporated herein by reference in its entirety. For example, the bladder 98 may be positioned between the cords (i.e. zip ties) of the device and the patient's body, to monitor the pressure applied to the patient's body.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. A wound management assembly comprising:
a belt configured to partially wrap around the body of a patient in the area of a wound;
first and second base members configured to contact the patient on opposing sides of the wound and to grip opposing end regions of the belt to permit engagement, tensioning and detensioning of the belt relative to the base members;
at least one cord for connecting the base members to each other across the wound, wherein the cord is adjustable to increase and decrease the pressure applied to the body of the patient; and
a pressure monitoring assembly for monitoring the pressure applied to the body of the patient, wherein the pressure monitoring assembly comprises a bladder positionable between the at least one cord and the body of the patient, and a pressure gauge for displaying a pressure associated with the bladder.

2. The wound management assembly of claim 1, wherein the bladder comprises an inflatable bag.

3. The wound management assembly of claim 1, wherein when uninflated, the bladder has a length of between about 5 inches and 15 inches.

4. The wound management assembly of claim 3, wherein when uninflated, the bladder has a width of between about 1 inch and about 3 inches.

5. The wound management assembly of claim 1, wherein when inflated, the bladder has a depth of between about 1 inch and about 1.5 inches.

6. The wound management assembly of claim 1, wherein the bladder comprises fasteners for securing the bladder to the cord.

7. The wound management assembly of claim 1, wherein the pressure gauge is configured to provide a pressure reading of between 0 inH₂O and 30 inH₂O.

8. The wound management assembly of claim 1, wherein the pressure gauge is coded to indicate a target pressure range to a user.

9. The wound management assembly of claim 7, wherein the pressure gauge is coded to indicate that the target pressure range is between about 20 inH₂O and 30 inH₂O.

10. The wound management assembly of claim 7, wherein the pressure gauge is coded to indicate that a pressure below 20 inH₂O is too low.

11. The wound management assembly of claim 7, wherein the pressure gauge is coded to indicate that a pressure above 27 inH₂O is too high.

12. The wound management assembly of claim 1, wherein the pressure gauge is securable to the belt, the first base member, or the second base member.

13. The wound management assembly of claim 11, wherein the pressure gauge is securable to belt with a hook-and-loop fastener.

14. The wound management assembly of claim 1, further comprising a pump for inflating the bladder.

15. The wound management assembly of claim 1, wherein the bladder is inflated by a fixed amount.

16. A method for wound management, comprising:
a. positioning first and second base members on a body of a patient on opposing sides of a wound;
b. placing a bladder on the body of the patient between the first and second base members;
c. positioning at least one cord over the bladder and connecting the base members to each other across the wound with the at least one cord;
d. partially wrapping a belt around the body of the patient in an area of the wound and gripping opposing end regions of the belt with the first and second base members; and
e. adjusting the at least one cord to increase or decrease the pressure applied to the body of the patient, whereby adjusting the at least one cord concurrently compresses or decompresses the bladder; and
f. reading a pressure gauge associated with the bladder to assess the pressure applied to the body of the patient.

17. The method of claim 16, wherein step e. comprises adjusting the at least one cord until the pressure gauge indicates that the pressure applied to the body of the patient is a target pressure.

18. The method of claim 16, wherein step e. comprises adjusting the at least one cord until the pressure gauge indicates a pressure of between 20 inH₂O and 27 inH₂O.

19. The method of claim 16, further comprising periodically repeating steps e. and f.

20. The method of claim 16, wherein step e. comprises adjusting the at least one cord to increase the pressure applied to the body of the patient if the pressure gauge indicates a pressure of below 20 inH₂O, and adjusting the at least one cord to decrease the pressure applied to the body of the patient if the pressure gauge indicates a pressure of above 27 inH₂O.
